(19) United States
(12) Patent Application Publication
(10) Pub. No.: US 2022/0401712 A1
(43) Pub. Date: Dec. 22, 2022

(54) AUTOMATED BODY FLUID DRAIN CONTROL APPARATUS WITH ONE OR MORE CAMERAS

(71) Applicant: BECKERSMITH MEDICAL, INC., Irvine, CA (US)

(72) Inventors: Jan M. Eckermann, Los Angeles, CA (US); Paul Intagliata, Long Beach, CA (US)

(21) Appl. No.: 17/843,453

(22) Filed: Jun. 17, 2022

Related U.S. Application Data
(60) Provisional application No. 63/297,121, filed on Jan. 6, 2022.
(60) Provisional application No. 63/211,903, filed on Jun. 17, 2021.

(51) Int. Cl. A61M 27/00; G06T 7/20; G06T 7/00; G06T 5/00; G06T 7/90

(52) U.S. Cl. CPC A61M 27/006; G06T 7/20; G06T 7/0016; G06T 5/001; G06T 7/90; A61M 2205/3306; A61M 2205/3334; A61M 2205/3313; A61M 2205/52; A61M 2205/18; A61M 2205/3344; G06T 2207/30004

(57) ABSTRACT

Cerebrospinal fluid (CSF) drainage systems. A system includes a conduit having a proximal end and a distal end. The conduit receives the CSF from a patient from the proximal end. The system includes a collection chamber coupled to the distal end. The collection chamber collects the CSF. The system includes a valve positioned on the conduit. The valve controls CSF flow into the collection chamber. The system includes a camera that captures an image of the CSF within the collection chamber. The system includes a processor coupled to the camera. The processor measures a flow rate of the CSF based on the image and controls the first valve to open for a first predetermined period and close for a second predetermined period until a determination of a predetermined amount of the CSF being drained from the patient is made by the processor based on the flow rate.

### CROSS-REFERENCE TO RELATED APPLICATIONS

This application claims the benefit of U.S. Provisional Application No. 63/211,903, filed on Jun. 17, 2021, and U.S. Provisional Application No. 63/297,121, filed on Jan. 6, 2022, the entireties of which are incorporated by reference herein.

### FIELD OF THE INVENTION

The present invention relates to an automated body fluid drain control apparatus.

### BACKGROUND OF THE INVENTION

Body fluid drains and containers are well known in the art. For example, there are collection devices for urine and others that drain and collect spinal fluid. None of these devices are able to easily control the drainage rate of the fluid as a function of time.

In connection with the drainage of cerebrospinal fluid (“CSF”), for most people, the body produces 450 ccs of CSF over a 24 hour period which fills the subarachnoid space in the body. There are many instances where it may be advisable and/or necessary for some of the CSF to be drained. For example, during certain medical procedures such as brain surgery, the surgeon may wish to drain some of the CSF in order to retract the brain. In addition, in some brain and spinal surgeries where the dura mater is penetrated, the CSF would need to be partially drained to keep pressure off the wound site in order to allow it to heal. Also, in certain head trauma cases where CSF is collecting in the cranial cavity, it may be preferable to drain some of the CSF from the subarachnoid space in the lumbar spinal region to relieve the pressure on the brain.

Conventional methods of draining CSF involve tapping into the cranial or subarachnoid space in the spinal column and draining the excess CSF through a catheter tube into a collection bag. The amount of drainage must be regulated, as if there is too much drainage, a patient can be irreversibly injured or can be fatally injured.

Unfortunately, the rate at which the CSF drains is not linear in fashion. For example, the CSF can drain at 1 cc per hour and then suddenly drain 5 ccs in 10 minutes. Since there are irreversible and potentially fatal consequences if too much CSF is drained, the volume of the drainage has to be constantly monitored by a nurse. Due to the demand on a nurse's time and the non-linearity of the drainage, there is a potentially fatal margin of error. Thus, an apparatus that continuously monitors and controls the drainage of the CSF would be of great benefit to the art. Moreover, patients having CSF fluid or other fluids drained are often bed ridden due to the size of the monitoring machines. Accordingly, the ability for a patient to be able to get up out of bed and move around while the fluid is still being drained is advantageous. Further, while normal or healthy CSF is clear, contaminated or abnormal CSF may present a variety of colors depending on the contaminant or the condition. As such, systems that detect and process the color of drained CSF to diagnose, assess, or observe patient condition would also be of great benefit to the art

The background description includes information that may be useful in understanding the present invention. It is not an admission that any of the information provided herein is prior art or relevant to the presently claimed invention, or that any publication specifically or implicitly referenced is prior art.

### SUMMARY OF THE PREFERRED EMBODIMENTS

The present invention allows the use of different light refraction properties to diagnose and allow for early prediction of certain pathologies within the cerebrospinal fluid (CSF) as well as regulate flow rate of CSF based on image detection and/or pressure readings. Any type of light refraction technology, such as spectrophotometry, can be utilized in the present invention and the device related thereto. A camera can also be utilized.

A preferred embodiment includes a CSF drainage system. The system includes a first conduit having a proximal end and a distal end. The first conduit is configured to receive the CSF from a patient from the proximal end. The system further includes a first collection chamber coupled to the distal end of the first conduit. The first collection chamber is configured to collect the CSF transported by the first conduit. The system further includes a first valve positioned on the first conduit. The first valve is configured to control flow of the CSF into the first collection chamber. The system further includes a camera. The camera is configured to capture an image of the CSF collected within the first collection chamber. The system further includes a processor coupled to the camera. The processor is configured to measure a flow rate of the CSF based on the image and control the first valve to open the first valve for a first predetermined period and close the first valve for a second predetermined period until a determination of a predetermined amount of the CSF being drained from the patient is made by the processor based on the measured flow rate.

This and other embodiments may optionally include the following. The processor may be further configured to close the first valve until an end of a third predetermined period when the predetermined amount of the CSF is drained from the patient before the third predetermined period has elapsed. The processor may be further configured to determine a count of pixels defining the CSF collected within the first collection chamber on the image to measure the flow rate of the CSF. The processor may be further configured to adjust one or more properties of the image to increase visibility of the pixels prior to determining the count of the pixels. The system may further include a second valve configured to allow the first collection chamber to collect the CSF in a closed position and drain the collected CSF in the first collection chamber in an open position. The processor may be further configured to open the second valve upon the determination of the predetermined amount of the CSF being drained from the patient. The system may further include a second conduit having a proximal end and a distal end. The second conduit may be configured to receive the CSF from the first collection chamber from the proximal end. The system may further include a second collection chamber coupled to the distal end of the second conduit. The second collection chamber may be configured to collect the CSF transported by the second conduit. The second valve may be positioned on the second conduit.

Another preferred embodiment includes a CSF drainage system. The system includes a conduit having a proximal end and a distal end. The conduit is configured to receive the CSF from a patient from the proximal end. The system further includes a collection chamber coupled to the distal end of the first conduit. The collection chamber is configured to collect the CSF transported by the first conduit. The system further includes a camera configured to capture an image of the CSF collected within the collection chamber. The system further includes a sensor coupled to the camera. The sensor is configured to determine a wavelength of a color of the CSF from the image. The system further includes a memory configured to store a database including wavelength data or wavelength range data and diagnostic data associated with each of the wavelength data or each of the wavelength range data. The system further includes a processor coupled to the camera, the sensor, and the memory. The processor is configured to determine a diagnosis, assessment, or a condition based on the wavelength of the color of the CSF and the database.

This and other embodiments may optionally include the following. The database may be compiled based on a plurality of images of the CSF collected within the collection chamber and the wavelength determinations of the colors of the CSF from the plurality of images over time. The system may further include an output device coupled to the processor and configured to output the trajectory of health condition of the patient. The output device may be further configured to output an alert or a notification associated with the diagnosis or the trajectory of health condition based on predetermined alert or notification settings. The diagnosis, assessment, or the condition may include an infection, a disease, or bleeding,

Yet another preferred embodiment includes a CSF drainage system. The system includes a first conduit having a proximal end and a distal end. The first conduit is configured to receive the CSF from a patient from the proximal end. The system further includes a first collection chamber coupled to the distal end of the first conduit. The first collection chamber is configured to collect the CSF transported by the first conduit. The system further includes a first valve positioned on the first conduit. The first valve is configured to control flow of the CSF into the first collection chamber. The system further includes a pressure sensor coupled to the first conduit. The pressure sensor is configured to measure intracranial pressure. The system further includes a processor coupled to the pressure sensor. The processor is configured to repeatedly open and close the first valve when the intracranial pressure is at or above a predetermined pressure for a predetermined period to reduce the intracranial pressure.

This and other embodiments may optionally include the following. The processor may be further configured to partially open the first valve at a predetermined percentage to regulate the intracranial pressure based on the measured intracranial pressure. The system may further include a second valve configured to allow the first collection chamber to collect the CSF in a closed position and drain the collected CSF in the first collection chamber in an open position. The processor may be further configured to open the second valve upon a determination that the intracranial pressure has reduced to a desired pressure value. The system may include a second conduit having a proximal end and a distal end. The second conduit may be configured to receive the CSF from the first collection chamber from the proximal end. The system may further include a second collection chamber coupled to the distal end of the second conduit. The second collection chamber may be configured to collect the CSF transported by the second conduit. The second valve may be positioned on the second conduit.

Spectrophotometry allows the device to detect impurities within the CSF such as hemoglobin (blood), white blood cells (indicative of infection), etc. The wavelengths are different depending on the CSF sample contents (e.g., based on the contaminants in the CSF).

A built-in microprocessor with a pre-programmed reference library of the range of wavelengths corresponding to different anomalies, contaminants and quantities thereof, etc. within the CSF allows the device to alert the user about the changes in CSF. Serial recording allows the user to trend pathologies. One such example is trending the amount of hemoglobin in the CSF to see if a brain bleed is resolving or worsening. Same for infections and other microbiological markers within the CSF such as markers for TBI, Alzheimer's, etc. If there is anything abnormal that is detected, e.g., by the spectrophotometer, the abnormal CSF values may be displayed on the user interface and also possibly in the user's medical records (e.g., via Bluetooth or other wireless connection or a wired connection). If an abnormality is detected (e.g., within a predetermined or pre-specified wavelength range), the device may emit a visual or audible signal.

In a preferred embodiment, the present invention is a CSF management platform that allows for therapeutic drainage and also as a diagnostic, assessment, or observation tool in real time. The device can measure drainage and can regulate the flow rate with respect to pressure. If the pressure goes up it will increase the outflow rate and if the pressure goes down it will decrease the outflow rate to keep the intracranial pressure within a desired or predetermined range. The real time capability allows drainage and pressure sensing at the same time.

The claim is that this is the first device that can perform this type of analysis in real time without having to send samples to the laboratory. In a preferred embodiment, instead of a spectrophotometer, a camera can be used to detect the relevant fluid levels and further to detect fluid contaminants and other changes within the fluid. The contaminants and other anomalies found within the fluid can be compared against a database of reference library of information based on different wavelengths to determine any issues with the patient.

In accordance with an aspect of the present invention there is provided a portable body fluid collection device that includes an apparatus for controlling the collection of body fluids and at least one strap associated with the apparatus for controlling the collection of body fluids. The strap can be worn by a patient to support the weight of the automated body fluid drain control apparatus. The apparatus for controlling the collection of body fluids includes a drainage tube and a fluid collection chamber in fluid communication with the drainage tube. The fluid collection chamber includes a first valve, and the fluid collection chamber is configured such that when a predetermined amount of fluid is collected in the collection chamber before a first predetermined period of time elapses, the collection chamber ceases collecting fluid. In a preferred embodiment, the apparatus for controlling the collection of body fluids further includes a measuring device (e.g., camera) that measures the amount of fluid entering the collection chamber through the first valve during the predetermined first period of time. The first valve has an open and a closed state. The first valve is normally open and allows body fluids to flow into the collection chamber. During the first period of time, if a predetermined volume of body fluid enters the chamber, the first valve is closed.

In a preferred embodiment, the apparatus for controlling the collection of body fluids further includes a second valve having an open and a closed state. The second valve is normally closed so that body fluids collect in the chamber, and the second valve can be opened to empty the collection chamber. Preferably, after the first period of time has elapsed, the first valve is opened and the second valve is closed and a second period of time starts. In this embodiment, the first period of time is equal to the second period of time. In a preferred embodiment, the portable body fluid collection device further includes a container. The apparatus for controlling the collection of body fluids is disposed in the container, and the strap is connected to the container.

In a preferred embodiment, the drainage tube is configured to be inserted into a patient's subarachnoid region at an insertion point. The container and strap are configured such that when the container is worn by a patient, the first valve is positioned below the insertion point. Preferably, the container is vertically adjustable between at least a first and a second position. Preferably, the container is a backpack.

In accordance with another aspect of the present invention there is provided a method of collecting body fluid that includes providing an apparatus for controlling the collection of body fluids, inserting an end of the drainage tube into a patient's subarachnoid region at an insertion point, and securing the apparatus for controlling the collection of body fluids to the patient's body at a first position, wherein in the first position, the first valve is located vertically below the insertion point. The apparatus for controlling the collection of body fluids includes a drainage tube, and a fluid collection chamber in fluid communication with the drainage tube. The fluid collection chamber includes a first valve, and the fluid collection chamber is configured such that when a predetermined amount of fluid is collected in the collection chamber before a first predetermined period of time elapses the collection chamber ceases collecting fluid. Preferably, the apparatus for controlling the collection of body fluids is secured to the patient's body via a strap. In a preferred embodiment, the strap is connected to a container, and at least a portion of the apparatus for controlling the collection of body fluids is disposed in the container.

In a preferred embodiment of the present invention, an automated fluid collection apparatus comprises a first tube having a first end connected to a drain that has been inserted into a patient's subarachnoid region and a second end connected to an opening at the proximal end of a first collection chamber. The first collection chamber also has an opening at the distal end thereof. In one embodiment the chamber has a first valve or first controllable closing mechanism proximate the opening at the proximal end and a second valve or controllable closing mechanism at the distal opening thereof. In another embodiment, there is a second tube having a first end and a second end, whereby the first end is connected to the opening at the distal end of the collection chamber. In the preferred embodiments of the present invention, the first and second valves may be located on the first and second tubes respectively or on the proximal and distal ends of the first chamber. In one preferred embodiment, the distal end is connected directly to a collection bag; in another embodiment the second tube is connected at the second end to a second collection chamber or bag.

In the preferred embodiment, the apparatus also comprises a measuring device which determines the amount of fluid that is being collected in the first chamber over a preselected period of time. In one preferred embodiment, the measuring device is a camera that is able to detect specific wavelengths inside the chamber so as to determine the amount of fluid collected herein. In other alternate embodiments, a different type of fluid measuring device may be used. In an alternate embodiment, the measuring device may also be able to sense the type of fluid collected in the chamber to detect any anomalies therein.

The apparatus also comprises a timer, microprocessor and power supply which are connected to the measuring device and to the controllable closing mechanisms so that the amount of fluid collected in the first collection chamber is constantly being measured, monitored and controlled. The microprocessor processes the various measurements received from the measuring device to determine the volume contained within the first chamber. The timer controls the period of time over which the fluid is measured and collected within the first chamber and is reset for each new period of time as instructed by the processor. Once the microprocessor determines that the first chamber is filled to a preselected level at any time prior to the expiration of the selected time period, it will cause the first valve to close and the second valve to open such that the first chamber will be emptied and so that the drainage will discontinue for the remainder of the preselected time period. In this manner, the drainage rate of the fluid will never be more than the preselected level during the preselected period of time.

In the preferred method of the present invention, the first end of the first tube is attached to a drain that has been inserted into the appropriate subarachnoid region of the body. The first valve is opened and the second valve is closed. The timer is also set for either a default or an alternate timer period as is the maximum volume for the first chamber.

Thereafter, the CSF fluid drains through the proximal valve into the first collection chamber through the use of gravity. As the fluid drains into the first chamber, the volume of the first chamber is constantly measured by the measuring device. Once the microprocessor determines that the first chamber is filled to a preselected level at any time prior to the expiration of the then applicable preselected time period, it will cause the first valve to close and the second valve to open such that the first chamber will be emptied and so that the drainage will discontinue for the remainder of the preselected time period. In this manner, the drainage rate of the fluid will never be more than the preselected level during the preselected period of time. At the end of each preselected time period the timer will reset.

If the first chamber has not attained the maximum preselected volume in the preselected time period, the first valve will remain open and the second valve will remain closed and the drainage will continue until the maximum volume is attained.

If at any time there is any problem with the system or if the first collection chamber fails to fully empty at each predetermined interval, an alarm will notify the appropriate personnel that their attention is required. See U.S. Patent Publication No. 2017/0304596 (the “'596 publication”), the entirety of which is incorporated herein by reference.

The invention, together with additional features and advantages thereof, may be best understood by reference to the following description.

### BRIEF DESCRIPTION OF THE DRAWINGS

FIG. 1 is a perspective view of a preferred embodiment of the apparatus of the present invention in a closed position;

FIG. 2 is a partial view of an alternate e embodiment of the apparatus of the invention in a closed position;

FIG. 3 is an exploded view of the preferred embodiment of FIG. 1 in an open position;

FIG. 4 is a perspective view of a portable version of the apparatus of the present invention that can be rolled;

FIG. 5 is a perspective view of a portable body fluid collection device in accordance with a preferred embodiment of the present invention;

FIG. 6 is a perspective view of the portable body fluid collection device of FIG. 5 adjusted to a lower position;

FIG. 7 is a perspective view of another portable body fluid collection device in accordance with a preferred embodiment of the present invention; and

FIG. 8 is a perspective view of another portable body fluid collection device in accordance with a preferred embodiment of the present invention.

FIG. 9 is a perspective view of a CSF drainage system in accordance with a preferred embodiment of the present invention;

FIG. 10 is a schematic of the system of FIG. 9;

FIG. 11 is a processed image of the CSF collected within a collection chamber of the system of FIG. 9;

FIG. 12 is a graph of exemplary flow rate control by the system of FIG. 9;

FIG. 13 is a graph of exemplary flow rate control by the system of FIG. 9;

FIG. 14 is a schematic of a CSF drainage system in accordance with a preferred embodiment of the present invention;

FIG. 15 is a perspective view of a CSF drainage system in accordance with a preferred embodiment of the present invention; and

FIG. 16 is a schematic of the system of FIG. 15.

Like numerals refer to like parts throughout the several views of the drawings.

### DETAILED DESCRIPTION OF THE PREFERRED EMBODIMENTS

The following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be, but not necessarily are references to the same embodiment; and, such references mean at least one of the embodiments. If a component is not shown in a drawing then this provides support for a negative limitation in the claims stating that that component is “not” present. However, the above statement is not limiting and in another embodiment, the missing component can be included in a claimed embodiment.

Reference in this specification to “one embodiment,” “an embodiment,” “a preferred embodiment” or any other phrase mentioning the word “embodiment” means that a particular feature, structure, or characteristic described in connection with the embodiment is included in at least one embodiment of the-disclosure and also means that any particular feature, structure, or characteristic described in connection with one embodiment can be included in any embodiment or can be omitted or excluded from any embodiment. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others and may be omitted from any embodiment. Furthermore, any particular feature, structure, or characteristic described herein may be optional. Similarly, various requirements are described which may be requirements for some embodiments but not other embodiments. Where appropriate any of the features discussed herein in relation to one aspect or embodiment of the invention may be applied to another aspect or embodiment of the invention. Similarly, where appropriate any of the features discussed herein in relation to one aspect or embodiment of the invention may be optional with respect to and/or omitted from that aspect or embodiment of the invention or any other aspect or embodiment of the invention discussed or disclosed herein.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted.

It will be appreciated that the same thing can be said in more than one way. Consequently, alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long,” “up,” “down,” “aft,” “forward,” “inboard,” “outboard” and “below” used herein are merely for ease of description and refer to the orientation of the components as shown in the figures. It should be understood that any orientation of the components described herein is within the scope of the present invention.

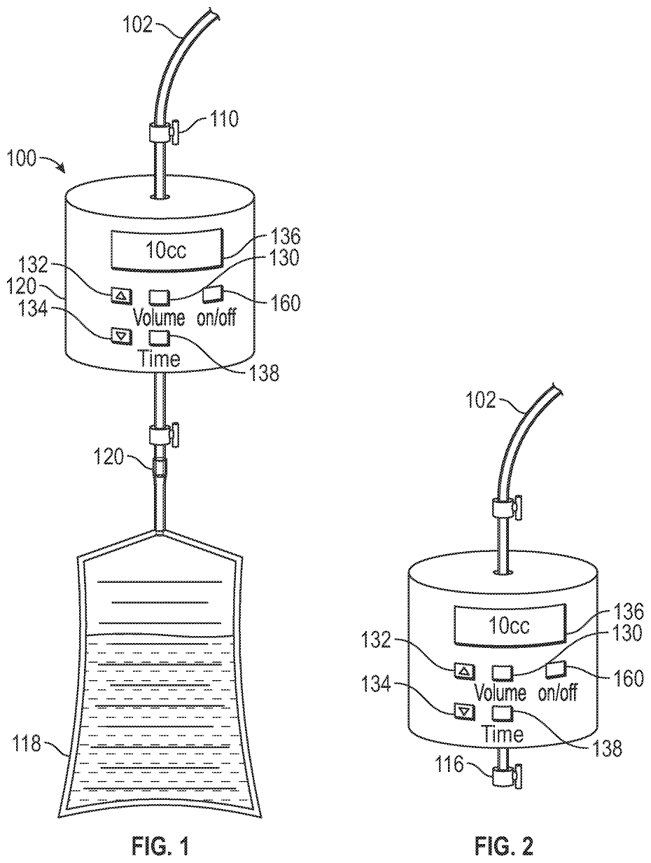

Generally, the present invention may be briefly described as follows. Referring first to FIGS. 1 and 2, a preferred embodiment of an automated fluid drain control apparatus 100 of the present invention is shown.

The automated fluid drain control apparatus 100 comprises a comprises a first tube 102 having a first end (not shown) connected to a drain (not shown) that has been inserted into a patient's subarachnoid region and a second end 104 connected to an opening 106 at the proximal end of a first collection chamber 108. In a preferred embodiment, the first collection chamber 108 has a first valve 110 or other suitable first controllable closing mechanism known in the art that is capable of opening and closing the flow of fluid between first tube 102 and the first collection chamber 108. In the preferred embodiments shown in FIGS. 1, 2 and 3, valve 110 is located on tube 102. However, in an alternate embodiment, not shown, there is no valve 110. In the embodiment shown, valve 110 is a manual valve that can be opened and closed by the user of the apparatus. However, in alternate embodiments, the valve 110 can be controlled electronically.

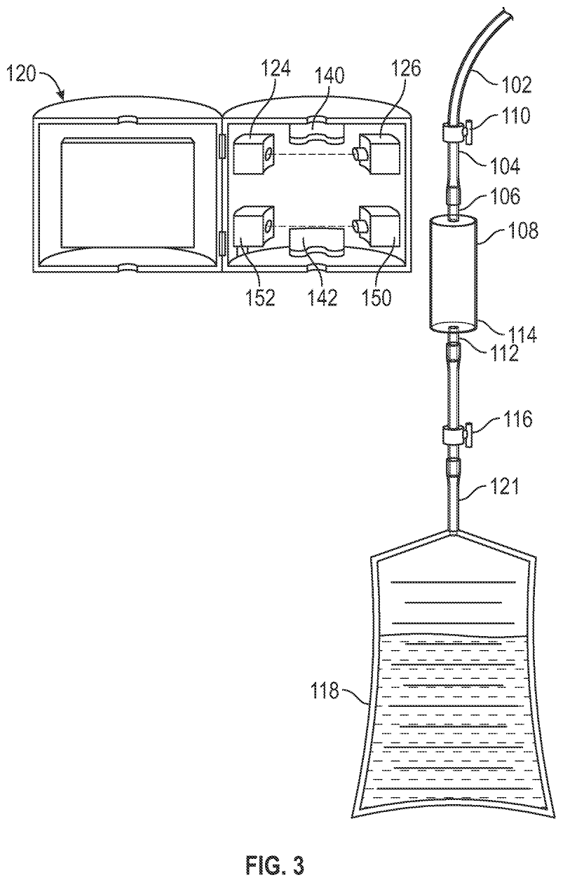

The first collection chamber 108 also has an opening 112 at the distal end 114 thereof. In FIG. 2, an alternate embodiment is shown in which the first collection chamber 108 has a second valve 116 or other suitable second controllable closing mechanism known in the art that is capable of opening and closing the flow of fluid out of the distal end 114 of the first collection chamber 108. In the embodiments shown in FIGS. 1 and 3, there is a second collection chamber 118 with a neck 121 which is connected to the opening 112 at the distal end 114 of the first collection chamber 108. In yet another embodiment (not shown) a second tube having a first end and a second end is connected at the first end to the opening 112 at the distal end 114 of the collection chamber 108. In that embodiment, the second tube is also connected at the second end to a second collection chamber or bag such as the one shown in FIGS. 1 and 3.

In a preferred embodiment, the apparatus 100 also includes a housing 120 which houses the first chamber 108 and the electronics (not shown) which include a microprocessor (not shown), a timer (not shown), a power supply (not shown) and relays (not shown) which control third and fourth valves 140 and 142. The third 140 and fourth 142 valves are either pinch valves or any other mechanism known in the art that are controllable and which can easily and quickly impede the flow of fluid. In addition, in a preferred embodiment shown in FIG. 3, the third and fourth valves 140 and 142 are located in the housing proximate the beginning of the first and second tubes so that they can impede the flow of fluid into and out of the first collection chamber 108. However, in alternate embodiments there is only one set of valves such that either valves 110 and 116 are controlled electronically such that there is no need for third and fourth valves or valves 110 and 116 are unnecessary. However, in order to provide a fail-safe mechanism, in the preferred embodiment, all four valves are used.

In addition, the housing 120 also contains a first measuring device connected to the microprocessor which together determine the amount of fluid that is being collected in the first chamber 108 over a preselected period of time. In one preferred embodiment, shown in FIG. 3 the measuring device is an optical sensor 124 connected to a camera or spectrophotometer 126 which is able to detect specific wavelengths by shining a light through the first collection chamber 108 so as to determine the amount of fluid collected therein. In other alternate embodiments, a different type of fluid measuring device may be used with suitable modifications to the electronics and microprocessor. By way of example, and not limitation, such measuring devices may include, a weight measuring device that measures the amount of fluid collected within the first collection chamber 108 based upon weight; an acoustic sensor coupled with a sound generator which sends sound waves through the chamber 108 in order to detect the amount of fluid based upon the change in the sound waves as they traverse the fluid; a capacitive sensor either located within the first collection chamber to measure a variable such, as, but not limited to, the pressure inside the chamber created by the changes in the volume of fluid to determine the volume or depending on the material of the chamber, detect and measure deformations of the chamber again to determine volume; a flowmeter; a thermo sensor; a ph sensor; a device that uses the technology of microfluidics; or any other fluid measuring device known in the art.

In various embodiments of the present invention with suitable additional software added to the microprocessor, the measuring device also is able to detect any anomalies such as, but not limited to, the presence of blood, white cells, pus, dye, etc.

The microprocessor is connected to the measuring device, the timer and the relays. In the embodiment shown in FIGS. 1-3, the relays are connected to the third and fourth valves 140 and 142. However, in an alternate embodiment in which there are no third and fourth valves, the relays are connected to the first and second valves 110 and 116.

The microprocessor ensures that the fluid inside the first collection chamber constantly is being measured, monitored and controlled. The microprocessor also processes the various measurements received from the measuring device to determine the volume contained within the first chamber.

In one embodiment, the user can select the maximum volume that can be collected in the first collection chamber 108 during a selected period of time. In the embodiment shown in FIGS. 1 and 2, that is accomplished by pressing the volume control button 130 located on the front of housing 120 and thereafter pressing up and down indicator buttons 132 and 134 which sends a signal to the microprocessor which then stores the amount of maximum volume selected in its memory. Alternate selections means well known in the art can be used in lieu of the buttons shown in FIGS. 1 and 2. In addition, in embodiments such as is shown in FIGS. 1 and 2, a visual display 136 is shown on the front of the housing 120 which shows the volume amount selected.

In another embodiment, the microprocessor selects the default value for the amount of volume that can be selected during a selected period of time. Regardless of how the volume is selected, the volume value regulates how many times the chamber will empty per preselected time period.

In another embodiment, the user can select the period of time over which the fluid is measured and collected within the first chamber. In the embodiment shown in FIGS. 1 and 2, that is accomplished by pressing the timer control button 138 located on the front of housing 120 and then up and down indicator buttons 132 and 134 which sends a signal to the microprocessor which then stores the amount of time selected in its memory. In addition, in an embodiment such as is shown in FIG. 1, the visual display 136 is shown on the front of the housing 120 which shows the timer amount selected. This also will regulate how many times the chamber will empty per preselected time period. If the timer is not manually set by the user, then the timer will set a default for the preselected time period. In another embodiment, which does not have a manual controller, the timer may be preselected by the microprocessor. In yet a further embodiment, the apparatus provides either means of selecting the period of time.

Regardless of the embodiment used, once the microprocessor determines that the first chamber is filled to a preselected level at any time prior to the expiration of the then applicable preselected time period, it will cause the appropriate valve to close which in the preferred embodiment shown in FIG. 3 is third valve 140 and the appropriate other valve to open which in the preferred embodiment shown in FIG. 3 is the fourth valve 142. In alternative embodiments in which only valves 110 and 116 are being used, then the microprocessor will cause first valve 110 to close and second valve 116 to open. In this manner, the first collection chamber 108 will be emptied and drainage will discontinue for the remainder of the preselected time period. As a result, the drainage rate of the fluid will never be more than the preselected level during the preselected period of time.

In addition, in various embodiments, the microprocessor also can determine and display the total volume drained over a larger time period on display 136 by holding down the volume button for a preselected period of time, although other embodiments may use other techniques well known in the art for obtaining the information with suitable modifications of the electronics.

In addition, to the foregoing, the apparatus may also contain an additional camera or spectrophotometer 150 and related sensor 152 to assure the complete emptying of the first collection chamber at the end of each appropriate cycle. In addition, the apparatus may also contain an alarm producing mechanism (not shown) whereby if the information sent by the measuring device as processed by the microprocessor detects that blood or other types of fluid or cells are present in the fluid, the nursing staff will be alerted. Likewise, with suitable additional electronics, other alerts may be present such as when there is a kink in the tubing, the power fails, or one of the components of the system is not operating properly, or if the first chamber fails to completely empty as required.

In the preferred method of the present invention, the first end of the first tube 102 is attached to a drain (not shown) that has been inserted into the appropriate subarachnoid region of the body. The appropriate valves associated with the proximal opening 106 of the first collection chamber 108 are opened and the appropriate valves associated with the distal end 114 of the collection chamber 108 are closed. In embodiments using four valves such as those shown in FIG. 3, the first and third valves 110 and 140 are opened and the second and fourth valves 116 and 142 are closed. The timer is also set for either a default or an alternate timer period and the maximum volume for the first chamber is also selected.

Thereafter, the fluid drains through the proximal valve(s) into the first collection chamber 108 through the use of gravity. As the fluid drains into the first chamber 108, the volume of the first chamber is constantly measured by the measuring device. Once the microprocessor determines that the first chamber 108 is filled to a preselected volume at any time prior to the expiration of the then applicable preselected time period, it will cause the proximal valve(s) to close and the distal valve(s) to open such that the first chamber will be emptied and the drainage will discontinue for the remainder of the preselected time period. In this manner, the drainage rate of the fluid will never be more than the preselected level during the preselected period of time. At the end of each preselected time period the timer will reset.

If the first chamber has not attained the maximum preselected volume in the preselected time period, the proximal valve(s) will remain open and the distal valve(s) will remain closed and the drainage will continue until the maximum volume is attained.

If at any time there is any problem with the system or if the first collection chamber fails to fully empty at each predetermined interval, an alarm will notify the appropriate personnel that their attention is required.

In addition, in the embodiments shown in FIGS. 1 and 2, the housing has an on/off button 160. Further, in the preferred embodiments, the tubing, bags, collection chamber and first and second valves are all disposable and replaceable. Those skilled in the art will understand that this type of apparatus is designed for use with CSF but it can be used in any other application in which the drainage amount of fluids over time is critical.

In another embodiment, the automated fluid drain control apparatus 100 includes a redundant fluid measuring system.

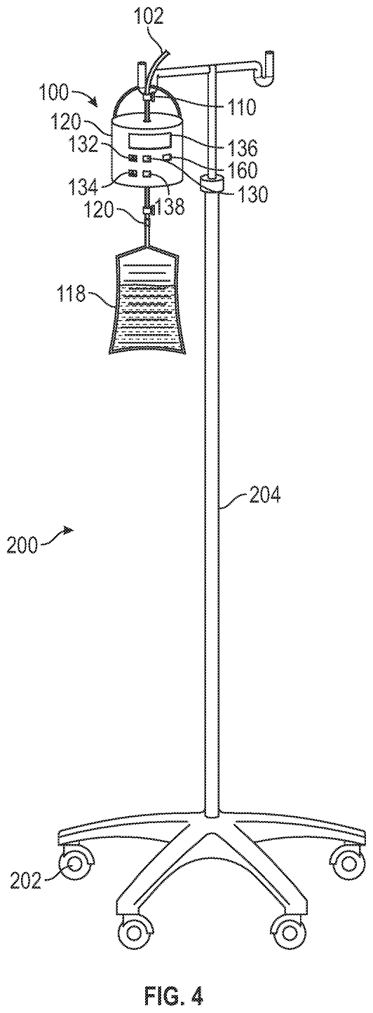

As shown in FIGS. 4-8, in other embodiments, the automated fluid drain control apparatus 100 is portable. As described above, in the prior art, close monitoring of patients having CSF drained is necessary. This confined a patient to bed. However, with the present automated system, a patient does not have to be as closely monitored by hospital personnel. Accordingly, the automated fluid drain control apparatus can be portable. For example, as shown in FIG. 4, in an embodiment of the invention, the automated fluid drain control apparatus 100 can be part of a tower assembly 200 that includes wheels 202. The apparatus 100 can be mounted on a tower 204 via a strap or permanently affixed thereto.

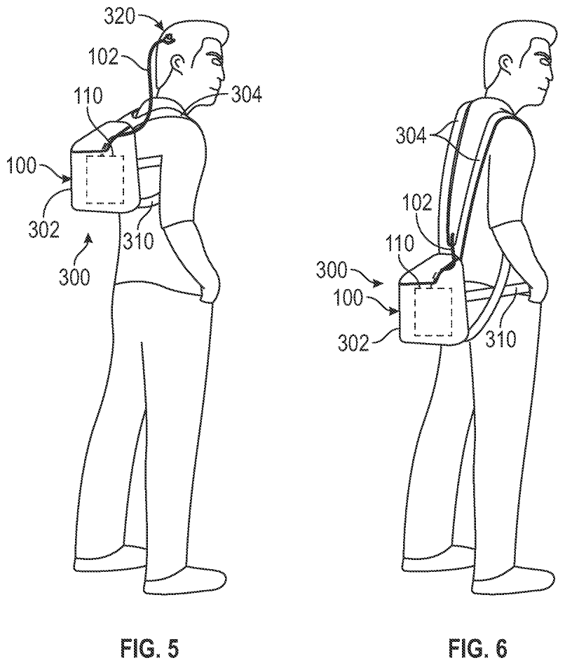

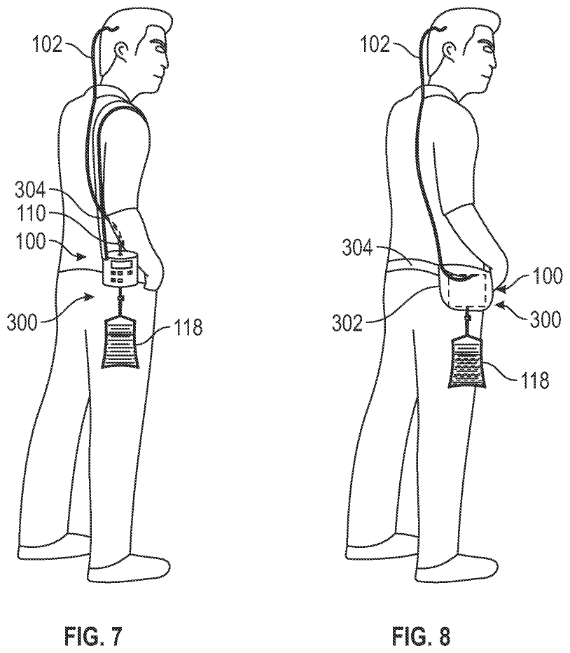

FIGS. 5-6 show a preferred embodiment of a portable body fluid collection device 300, that includes an apparatus for controlling the collection of body fluids, similar to that described above, drainage tube 102 and a container 302 for supporting or carrying the apparatus 100. As shown in FIGS. 5-6, in a preferred embodiment, the container 302 includes a first set of straps 304 that can be worn over the patient's shoulders, similar to a backpack. Preferably, the straps 304 are adjustable. In a preferred embodiment, the device also includes a second set of straps 310 that are worn around the front of the patient and clipped, clasped, tied or otherwise secured together. Preferably, straps 310 are also adjustable. It will be appreciated that the container 302 can be any device for carrying or supporting the apparatus 100. For example, the container 302 can be a bag, sack, box, shelf, purse, etc. In another preferred embodiment, the container 302 is a jacket into which the apparatus 100 is incorporated. Any type of wearable garment or container that can support the weight of the apparatus 100 and allow the tube 102 to extend into the subarachnoid space is within the scope of the present invention. For example, see the backpack taught in U.S. Publication No. 2012/0085804, published Apr. 12, 2012, the entirety of which is incorporated by reference.

As shown in FIG. 7, in another embodiment, one or more straps 304 can be connected directly to the apparatus 100 and the container 302 can be omitted. In this embodiment, the strap(s) 304 can be worn by a patient to support the weight of the apparatus 100. For example, as shown in FIG. 7, the strap 304 can be worn like a purse strap so that the apparatus 100 hangs near the patient's side.

In use, the drainage tube 102 (which may extend through an opening in the container) is inserted into the patient's subarachnoid region at an insertion point. The container 302, straps 304 and apparatus 100 are configured and positioned such that the first valve 110 is positioned below the insertion point, thereby allowing fluid to drain into the collection chamber via gravity. In a preferred embodiment, one or both of the container 302 or the straps 304 are adjustable in at least a vertical direction so that the first valve 110 can always be positioned below a chosen insertion point. FIG. 5 shows the positioning of the adjustable height container 302 when the tube 102 is inserted into an insertion point in the patient's skull. FIG. 6 shows the positioning of the adjustable height container 302 when the tube 102 is inserted into an insertion point in the patient's lower spine.

As shown in FIG. 8, in another embodiment, the container 302 and strap 304 can be formed as a fanny or hip pack. In this embodiment, the container 302 can be positioned on the patient's side making the apparatus 100 and the first collection chamber 108 or the second collection chamber 118 more accessible. In the embodiment shown in FIG. 8, the second collection chamber 118 or collection bag is located outside the container 302 so that fluid can be drained more easily. Therefore, the container 302 can have an opening (e.g., with a zipper, buttons, Velcro, etc.) that the container (and any associated tubing) extends through. However, in another embodiment, the collection bag can be located in the container 302. Similarly, the back pack embodiment above can include an opening therein that allows the collection bag to hang outside the container.

As shown in FIG. 5, in another embodiment, the portable body fluid collection device 300 can be combined with a device 320 that signals to a user when the tube 102 is being pulled. Co-pending and simultaneously filed patent application Ser. No. 14/209,983, filed Mar. 13, 2014 and titled Fluid Drain Tube with Connector (attorney docket no. 69638-5010), the entirety of which is incorporated by reference herein, describes a device 320 that pulls on a user's hair or skin when the tube 102 is pulled in. Device 320 can be combined with portable body fluid collection device 300 and sold as a kit. In this manner, with a person wearing the portable body fluid collection device 300, when they are moving around, device 320 will signal to the person that the tube 102 is moving and may help prevent withdrawal of the tube.

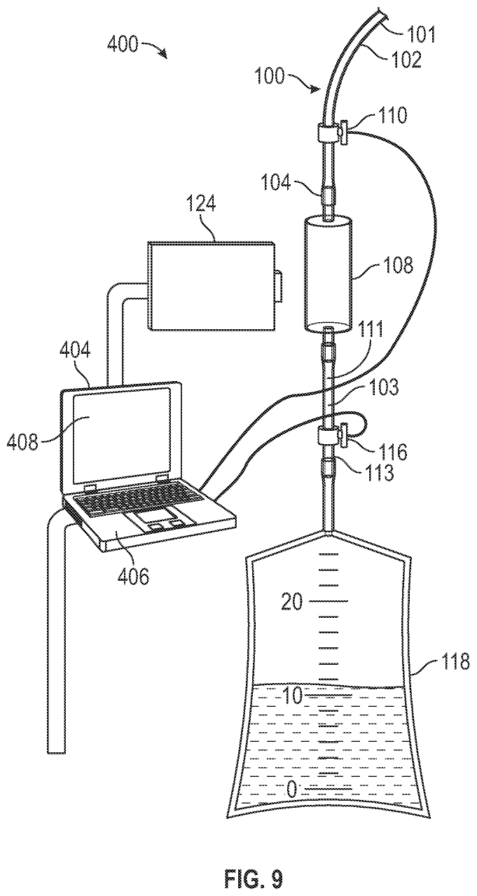

FIG. 9 is a perspective view of a CSF drainage system 400. The system 400 may include the automated fluid drain control apparatus 100 (see FIGS. 1-3), the housing 120 (see FIG. 3), and/or other embodiments discussed herein. The system 400 may include a camera 124 to capture an image of the CSF collected in the first collection chamber 108. In some examples, one or both of components 126 and 150 shown in FIG. 3 may be the camera. The image captured by the camera 124 may be used by the system 400 to measure CSF flow rate and changes in CSF flow rate. In some examples, the camera 124 may be coupled with a light source that is directed through the fluid so that the device does not have to depend on ambient lighting.

The apparatus 100 may have a first conduit or a first tube 102. The first tube 102 may have a proximal end or a first end 101 and a distal end or a second end 104. The first tube 102 may receive the CSF from a patient from the first end 101.

The apparatus 100 may have a first collection chamber 108. The first collection chamber 108 may be coupled to the second end 104 of the first tube 102. The first collection chamber 108 may receive and collect the CSF transported by the first tube 102. The CSF may be transported via gravity or a pump.

The apparatus 100 may have a first valve 110 positioned on the first tube 102. The first valve 110 may control flow of the CSF into the first collection chamber 108.

The apparatus 100 may have a second conduit or tube 103. The second tube 103 may have a proximal end 111 and a distal end 113. The second tube 103 may receive the CSF from the first collection chamber 108 from the proximal end 111.

The apparatus 100 may have a second valve 116 positioned on the second tube 103. The second valve 116 may allow the first collection chamber 108 to collect the CSF in a closed position. The second valve 116 may drain the collected CSF in the first collection chamber in an open position.

The apparatus 100 may have a second collection chamber 118. The second collection chamber 118 may be coupled to the distal end 113 of the second tube 103. The second collection chamber 118 may collect the CSF transported by the second tube 103 from the first collection chamber 108.

The apparatus 100 may include the camera 124. The camera 124 may be a conventional camera, such as a digital camera. The camera 124 may be positioned relative to the first collection chamber 108 such that the camera 124 can capture an entirety of the first collection chamber 108 in an image. In some examples, the camera 124 may be propped on a tripod or a similar device. In other examples, the camera 124 may be on or in the housing 120 (see FIG. 3).

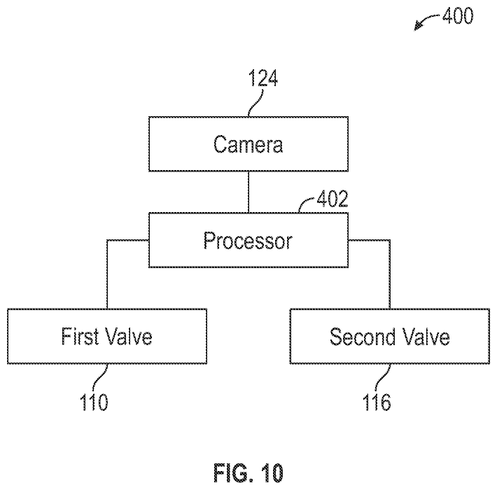

The system 400 may include a processor 402 (see FIG. 10). The processor 402 may be integrated with the camera 124 or another component of the apparatus 100. In some examples, the processor 402 may be a processor of a remote server in electronic communication with the system 400. In FIG. 9, the processor 402 is a processor of a computing device 404. The processor 402 may be configured to execute machine-readable instructions. In some examples, there may be a plurality of processors 402. In some examples, the processor 402 may be a microprocessor or a microcontroller. The processor 402 may be electronically coupled to the camera 124, wirelessly or wired.

The computing device 404 may have an input device 406 and an output device 408. The input device 406 may include buttons and/or a touchscreen. In other examples, the input device 406 may include knobs, dials, keys, pads, a mouse, a microphone, a camera, and/or the like. The input device 406 may be used to provide user instructions to the processor 402. The output device 408 may include a display, speakers, a haptic feedback motor, and/or the like. The output device 408 may present a user interface and/or data.

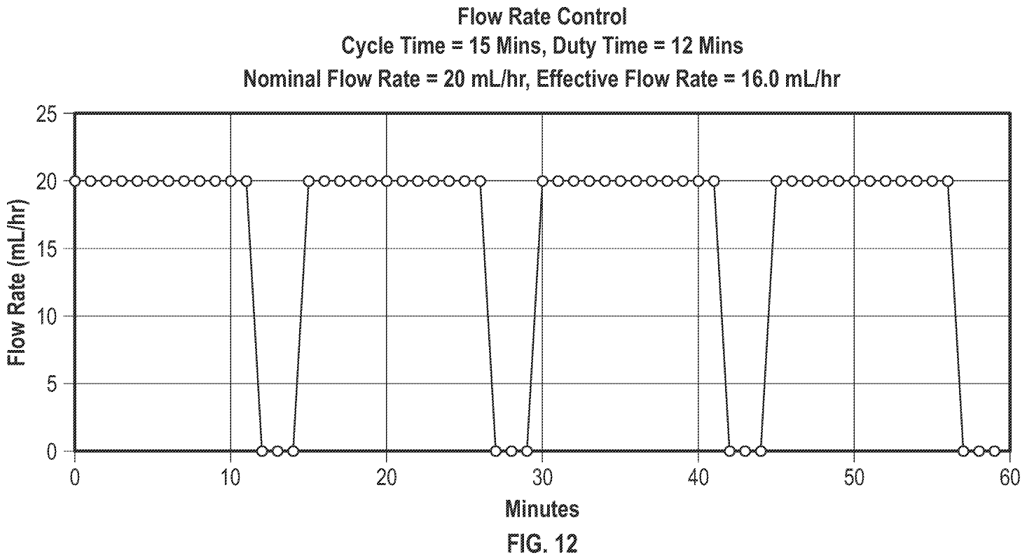

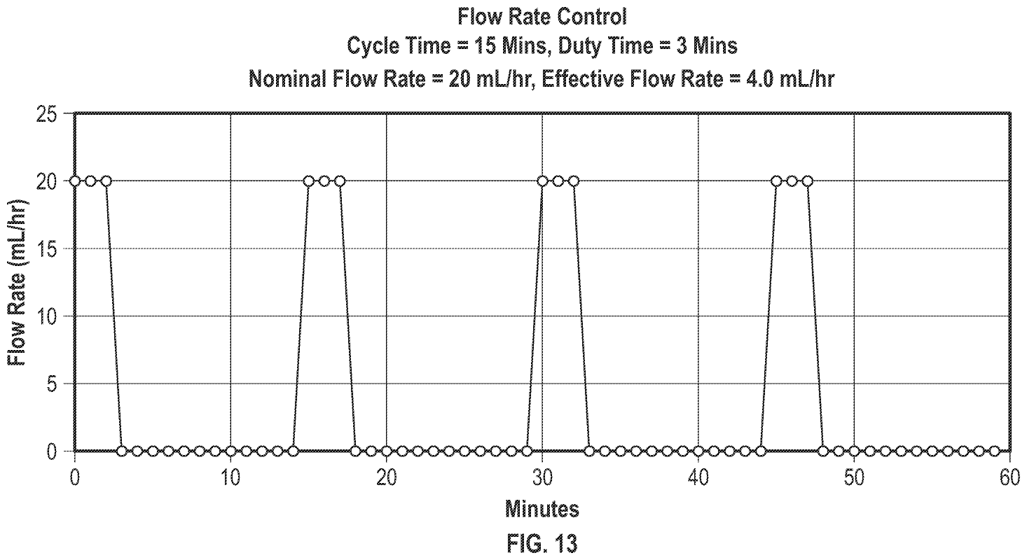

The processor 402 may be programmed to measure a flow rate of the CSF based on an image of the CSF collected within the first collection chamber 108 captured by the camera 124. The processor 402 may be further programmed to control the first valve 110 and the second valve 116 to open and close. For example, the processor 402 may open the first valve 110 for a first predetermined period and close the first valve 110 for a second predetermined period. The first and second predetermined periods may be adjusted by the user. For example, if a user desires a duty cycle of 100% within an hour, the first valve 110 may be open for the entire hour. In another example shown in FIG. 12, the duty cycle is 80% where the first valve 110 is repeatedly open for 12 minutes and closed for 3 minutes for an hour. Yet in another example shown in FIG. 13, the duty cycle is 20% where the first valve 110 is repeatedly open for 3 minutes and closed for 12 minutes for an hour. In other examples the duty cycle may be anywhere between and including 0% and 100%. The duty cycle may be increased if increased flow rate is desired and decreased if decreased flow rate is desired.

The processor 402 may be further programmed to determine whether a predetermined amount of CSF is drained from the patient based on the measured flow rate. The predetermined amount may be decided by a doctor, a medical professional, or the like. For example, the predetermined amount may be 10 cc (cubic centimeter). The processor 402 may be programmed to open and close the first valve 110 until the processor 402 determines that the predetermined amount of CSF has been drained from the patient.

The processor 402 may be further programmed to close the first valve 110 until an end of a third predetermined period when the predetermined amount of the CSF is drained from the patient before the third predetermined period has elapsed. The third predetermined period may be set by a doctor, a medical professional, or the like. For example, the third predetermined period may be an hour. The duty cycle may run during a course of the third predetermined period.

The processor 402 may be further programmed to open the second valve 116 upon the determination of the predetermined amount of the CSF being drained from the patient. Opening the second valve 116 will drain the first collection chamber 108. After the first collection chamber 108 is drained, the system 400 may restart the duty cycle or start a new duty cycle.

FIG. 10 is a schematic of the system 400. The camera 124 may be electronically coupled, wired or wirelessly, to the processor 402. The processor 402 may be electronically coupled, wired or wirelessly, the first tube 102 and the second tube 103. When the camera 124 captures an image of the CSF collected in the first collection chamber 108, the image may be transmitted to the processor 402 for processing. The camera 124 may periodically capture images. The processor 402, a processor of the camera 124, or a processor of a remote server may be programmed to continuously capture an image at a predetermined frequency. For example, the camera 124 may capture an image throughout a duty cycle, the third predetermined period, or until a predetermined amount of the CSF is drained from the patient.

The processor 402 may be programmed to adjust one or more properties of the image. The adjustment of the properties may increase visibility of the pixels defining or making up the CSF on the image. The properties that may be adjusted may include dimensions, field (e.g., masking, cropping), brightness, contrast, color plane, color scale, noise, and/or the like. For example, the processor 402 may mask the image to reduce the field to focus on the collected CSF. The processor 402 may then adjust the brightness of the image to make the pixels more visible. The processor 402 may then extract color planes to reduce the image to a single color plane. For example, the processor 402 may reduce the image to blue out of red, blue, and green. The processor 402 may then remove noise from the image and dilate a portion of the image. The portion may be where the collected CSF is shown. The processor 402 may the assign a threshold value for the pixels defining the CSF and return pixels set to the threshold value on a grayscale version of the image. The processor 402 may then remove unnecessary components of the image. The processor 402 may perform these adjustment in any sequence or simultaneously.

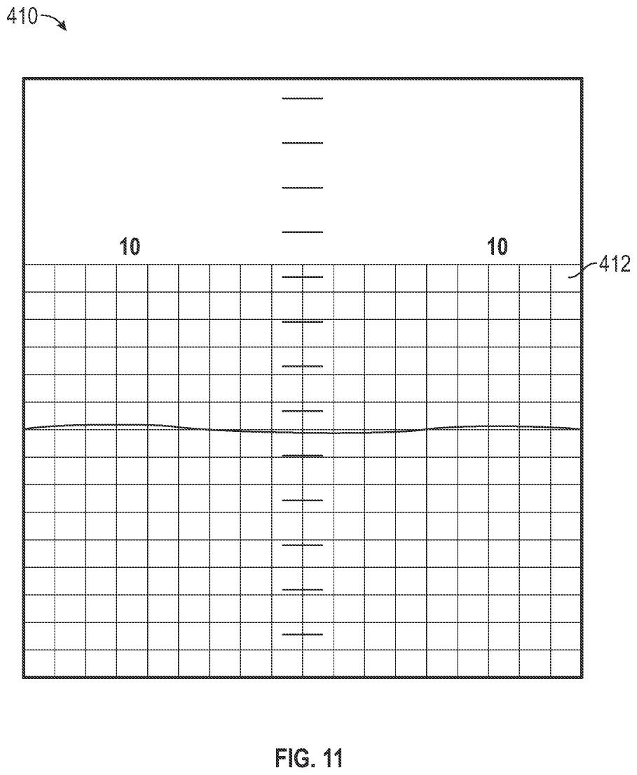

FIG. 11 is a processed image 410 of the CSF collected within the first collection chamber 108 (see FIG. 9) of the system 400 (see FIG. 9). The processed image 410 may show pixels 412 defining the CSF. The processor 402 may determine a count of the pixels 412. The count of the pixels 412 may provide a fluid level or volume. For example, the processed image shows a volume of 10 ccs. The processor 402 may find the volume of the CSF for one or more additional images. Then, the processor 402 may compare these images to determine a change over time to yield a flow rate of the CSF. The flow rate may then be used to control the first valve 110 and/or the second valve 116.

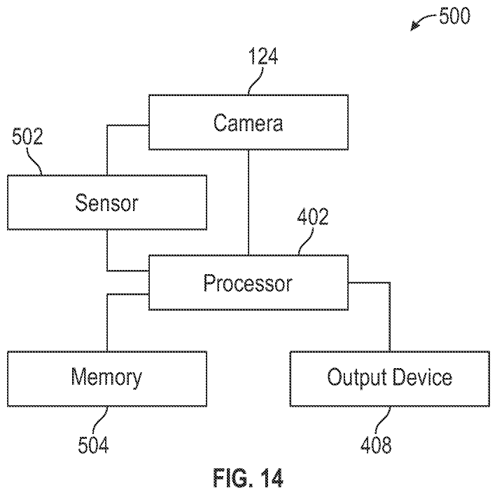

FIG. 14 is a schematic of a CSF drainage system 500. The system 500 may have the same specifications of the system 400 (see FIG. 9) or any other embodiment discussed herein. The system 500 may include a sensor 502 and a memory 504 in addition to the camera 124, the processor 402, and the output device 408. The camera 124, in conjunction with the sensor 502, may be used to detect the different wavelengths and to provide information as to the content of the CSF such as contaminants, infections, hemorrhage, proteins, biomarkers, etc. The system 500 also allows the use of different light refraction properties to diagnose and allow for early prediction of certain pathologies within the CSF. Any type of light refraction technology, such as spectrophotometry, may be utilized in the system 500 and the embodiments related thereto. However, in a preferred embodiment, the camera 124 is used.

The camera 124 allows the device to detect impurities within the CSF such as hemoglobin (blood), white blood cells (indicative of infection), neutrophils, macrophages, infected cells, particles, proteins, blood components, etc. The wavelengths are different depending on the CSF sample contents (i.e., based on the contaminants in the CSF).

The sensor 414 may be electronically coupled to the camera 124 and the sensor 502, wired or wirelessly. The sensor 414 may be a spectrophotometer. The sensor 414 may determine a wavelength of a color of the collected CSF within the first collection chamber 108. The sensor 414 may be integrated with the camera 124 in some examples. The camera 124 may transmit shade information to the sensor 414, which determines wavelengths within the shade information.

The memory 504 may be a random-access memory (RAM), a disk, a flash memory, an optical disk drive, a hybrid memory, or any other storage medium that can store data. The memory 504 may store program code that is executable by the processor 402. The memory 504 may store data in an encrypted or any other suitable secure form. In some examples, the memory 504 may be a memory of a remote server. In some examples, the memory 504 may be a memory of the computing device 404 or the camera 124.

The memory 504 may store a database that includes wavelength data and/or wavelength range and diagnostic data associated with each of the wavelength data or each of the wavelength range data. The range of wavelengths may correspond to different anomalies, contaminants and quantities thereof, etc. within the CSF, which allows the system 500 to transmit an alert about the changes in CSF.

The processor 402 may be electronically coupled, wired or wirelessly, to the camera 124, the sensor 502, the memory 504, and the output device 408. The processor 402 may be programmed to determine a diagnosis, assessment, or a condition based on the wavelength of the color of the CSF and the database. The diagnosis, assessment, or condition may be that the wavelength is within a normal range, meaning no blood contamination and no color or below a predetermined range of blood. The output device 408 may output the diagnosis, assessment, or the condition. The diagnosis, assessment, or condition may then be reviewed by the patient, the doctor, the medical staff, and/or the like. In some embodiments, the output device 408 may output an alert or a notification associated with the diagnosis, assessment, or condition based on predetermined alert or notification settings. For example, the output device 408 may display a visual, emit a sound, or vibrate as an alert or a notification. Predetermined alert or notification settings may include only producing an alert when the diagnosis, assessment, or condition is critical, severe, or urgent.

In a preferred embodiment, predetermined numbers are assigned to different levels of specific issues or the wavelengths related to the specific issues. The numbers will provide information to the clinician as to the seriousness or level of the issue. For example, a 1 can be assigned to a low level of blood/hemoglobin within the fluid (e.g., 0.0001 parts per million), and a 10 can be assigned to a level of blood/hemoglobin within the fluid that is an emergency (with other levels for 2-9 therebetween). These numbers or indices can provide the clinician guidance on which direction the status of the fluid is going (e.g., there is more or less blood in the fluid than yesterday).

The camera 124 may periodically capture images of the CSF collected within the first collection chamber 108 (see FIG. 9). The sensor 502 may determine a wavelength of the color of the CSF in each image. The processor 402 may be programmed to determine a diagnosis, assessment, or condition for each image based on the wavelength of the color of the CSF in each image. The processor 402 may be further programmed to compare the diagnoses, assessments, or observations of the images to each other to determine a trajectory of health condition of the patient. An algorithm can be used to check the wavelengths or level of contamination within the fluid against the database to give a prognostic value with regards to if the patient's condition has changed either improving or worsening. The trajectory of health condition may be outputted by the output device 408 similar to the output of the diagnosis, assessment, or condition and the alerts/notifications. The trajectory of health condition may include improving, worsening, no change, or stable statuses. The trajectory may be determined periodically (e.g., hourly, daily, on a minute basis).

Serial image capture and processing allows the user to trend pathologies. One such example is trending the amount of hemoglobin in the CSF to see if a brain bleed is resolving or worsening. Same for infections and other microbiological markers within the CSF such as markers for TBI, Alzheimer's (e.g., tau protein, neurofibrillary tangles), etc. If there is anything abnormal that is detected, e.g., by the camera 124 and/or the sensor 502, the abnormal CSF values may be displayed on the user interface and also possibly in the user's medical records (e.g., via Bluetooth®, a wireless connection, a wired connection). If an abnormality is detected (e.g., within a predetermined or pre-specified wavelength range), the output device 408 may emit a visual, audible, and/or haptic signal.

The database may be compiled in the memory 504 based on multiple images of the CSF collected within the first collection chamber 108 (see FIG. 9) and the wavelength determinations of the colors of the CSF from the multiple images over time.

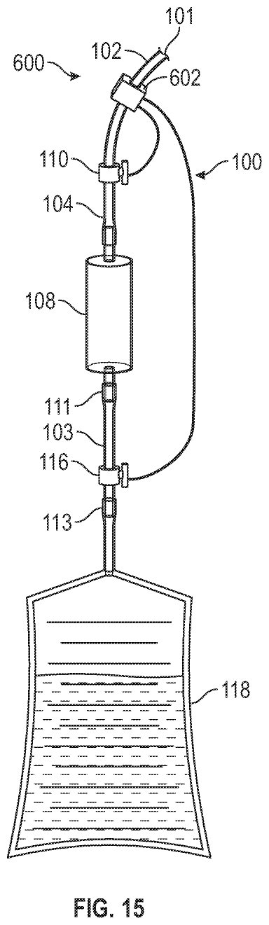

FIG. 15 is a perspective view of a CSF drainage system 600. The system 600 may have the same specifications of the system 400 (see FIG. 9), the system 500 (see FIG. 14) or any other embodiment discussed herein. The system 600 may additionally include a pressure sensor 602. The pressure sensor 602 may be coupled to the first tube 102. The pressure sensor 602 may measure intracranial pressure. The system 600 can measure drainage and can regulate the flow rate with respect to pressure. If the pressure goes up, the system 600 will increase the flow rate, and if the pressure goes down, it will decrease the flow rate to keep the intracranial pressure within a desired or predetermined range. For example, an intracranial that is less than 20 mmHg may be a desired or normal range. The real time capability allows drainage and pressure sensing at the same time. As such, the system 600 may advantageously perform the analysis in real time without having to send samples to the laboratory.

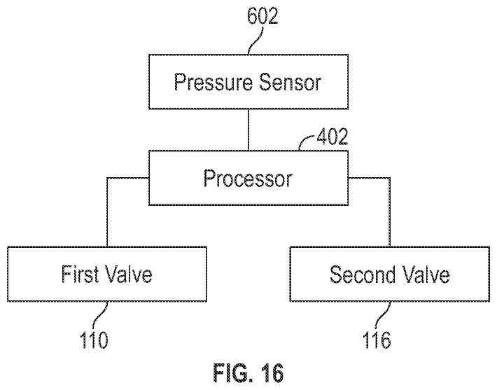

FIG. 16 is a schematic of the system of FIG. 15. The processor 402 may be electronically coupled, wired or wirelessly, to the pressure sensor 602. The processor 402 may be programmed to repeatedly open and close the first valve 110 when the intracranial pressure is at or above the predetermined pressure for a predetermined period (e.g., 20 minutes) to reduce intracranial pressure. In some examples, the processor 402 may be programmed to partially open the first valve 110 at a predetermined percentage (e.g., quarter open, half open, 75% open) to regulate the intracranial pressure based on the measured intracranial pressure.

The processor 402 may be further programmed to open the second valve 116 upon the system 600 determining that the intracranial pressure has reduced to a desired pressure value (e.g., below 20 mmHg). As such, the CSF collected within the first collection chamber 108 may be discharged. The discharge may be collected by the second collection chamber 118 through the second tube 103.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description of the Preferred Embodiments using the singular or plural number may also include the plural or singular number respectively. The word “or” in reference to a list of two or more items, covers all of the following interpretations of the word: any of the items in the list, all of the items in the list, and any combination of the items in the list.

The above-detailed description of embodiments of the disclosure is not intended to be exhaustive or to limit the teachings to the precise form disclosed above. While specific embodiments of and examples for the disclosure are described above for illustrative purposes, various equivalent modifications are possible within the scope of the disclosure, as those skilled in the relevant art will recognize. Further, any specific numbers noted herein are only examples: alternative implementations may employ differing values, measurements or ranges.

Although the operations of any method(s) disclosed or described herein either explicitly or implicitly are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

The teachings of the disclosure provided herein can be applied to other systems, not necessarily the system described above. The elements and acts of the various embodiments described above can be combined to provide further embodiments. Any measurements or dimensions described or used herein are merely exemplary and not a limitation on the present invention. Other measurements or dimensions are within the scope of the invention.

Any patents and applications and other references noted above, including any that may be listed in accompanying filing papers, are incorporated herein by reference in their entirety. Aspects of the disclosure can be modified, if necessary, to employ the systems, functions, and concepts of the various references described above to provide yet further embodiments of the disclosure.

These and other changes can be made to the disclosure in light of the above Detailed Description of the Preferred Embodiments. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features or aspects of the disclosure with which that terminology is associated. In general, the terms used in the following claims should not be construed to limit the disclosures to the specific embodiments disclosed in the specification unless the above Detailed Description of the Preferred Embodiments section explicitly defines such terms. Accordingly, the actual scope of the disclosure encompasses not only the disclosed embodiments, but also all equivalent ways of practicing or implementing the disclosure under the claims.

While certain aspects of the disclosure are presented below in certain claim forms, the inventors contemplate the various aspects of the disclosure in any number of claim forms. For example, while only one aspect of the disclosure is recited as a means-plus-function claim under 35 U.S.C. §112, ¶6, other aspects may likewise be embodied as a means-plus-function claim, or in other forms, such as being embodied in a computer-readable medium. (Any claims intended to be treated under 35 U.S.C. §112, ¶6 will include the words “means for”). Accordingly, the applicant reserves the right to add additional claims after filing the application to pursue such additional claim forms for other aspects of the disclosure.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. A cerebrospinal fluid (CSF) drainage system, comprising:
a first conduit having a proximal end and a distal end, the first conduit configured to receive the CSF from a patient from the proximal end;
a first collection chamber coupled to the distal end of the first conduit and configured to collect the CSF transported by the first conduit;
a first valve positioned on the first conduit and configured to control flow of the CSF into the first collection chamber;
a camera configured to capture an image of the CSF collected within the first collection chamber; and
a processor coupled to the camera and configured to measure a flow rate of the CSF based on the image and control the first valve to open the first valve for a first predetermined period and close the first valve for a second predetermined period until a determination of a predetermined amount of the CSF being drained from the patient is made by the processor based on the measured flow rate.

2. The CSF drainage system of claim 1 wherein the processor is further configured to close the first valve until an end of a third predetermined period when the predetermined amount of the CSF is drained from the patient before the third predetermined period has elapsed.

3. The CSF drainage system of claim 1 wherein the processor is further configured to determine a count of pixels defining the CSF collected within the first collection chamber on the image to measure the flow rate of the CSF.

4. The CSF drainage system of claim 3 wherein the processor is further configured to adjust one or more properties of the image to increase visibility of the pixels prior to determining the count of the pixels.

5. The CSF drainage system of claim 1 further comprising a second valve configured to allow the first collection chamber to collect the CSF in a closed position and drain the collected CSF in the first collection chamber in an open position, and wherein the processor is further configured to open the second valve upon the determination of the predetermined amount of the CSF being drained from the patient.

6. The CSF drainage system of claim 5 further comprising a second conduit having a proximal end and a distal end, the second conduit configured to receive the CSF from the first collection chamber from the proximal end and a second collection chamber coupled to the distal end of the second conduit and configured to collect the CSF transported by the second conduit, wherein the second valve is positioned on the second conduit.

7. A cerebrospinal fluid (CSF) drainage system, comprising:
a conduit having a proximal end and a distal end, the conduit configured to receive the CSF from a patient from the proximal end;
a collection chamber coupled to the distal end of the first conduit and configured to collect the CSF transported by the first conduit;
a camera configured to capture an image of the CSF collected within the collection chamber;
a sensor coupled to the camera and configured to determine a wavelength of a color of the CSF from the image;
a memory configured to store a database including wavelength data or wavelength range data and diagnostic data associated with each of the wavelength data or each of the wavelength range data; and
a processor coupled to the camera, the sensor, and the memory, the processor configured to determine a diagnosis based on the wavelength of the color of the CSF and the database.

8. The CSF drainage system of claim 7 wherein the camera is further configured to periodically capture one or more subsequent images of the CSF collected within the first collection chamber, the sensor is further configured to determine the wavelength of the color of the CSF from the one or more subsequent images, and the processor is further configured to determine one or more subsequent diagnoses based on the wavelength of the color of the CSF from the one or more subsequent images and compare the diagnosis and the one or more subsequent diagnoses among themselves to determine a trajectory of health condition of the patient.

9. The CSF drainage system of claim 8 wherein the database is compiled based on a plurality of images of the CSF collected within the collection chamber and the wavelength determinations of the colors of the CSF from the plurality of images over time.

10. The CSF drainage system of claim 8 further comprising an output device coupled to the processor and configured to output the trajectory of health condition of the patient.

11. The CSF drainage system of claim 10 wherein the output device is further configured to output an alert or a notification associated with the diagnosis or the trajectory of health condition based on predetermined alert or notification settings.

12. The CSF drainage system of claim 8 wherein the diagnosis includes an infection, a disease, or bleeding,

13. A cerebrospinal fluid (CSF) drainage system, comprising:
a first conduit having a proximal end and a distal end, the first conduit configured to receive the CSF from a patient from the proximal end;
a first collection chamber coupled to the distal end of the first conduit and configured to collect the CSF transported by the first conduit;
a first valve positioned on the first conduit and configured to control flow of the CSF into the first collection chamber;
a pressure sensor coupled to the first conduit and configured to measure intracranial pressure; and
a processor coupled to the pressure sensor and configured to repeatedly open and close the first valve when the intracranial pressure is at or above a predetermined pressure for a predetermined period to reduce the intracranial pressure.

14. The CSF drainage system of claim 13 wherein the processor is further configured to partially open the first valve at a predetermined percentage to regulate the intracranial pressure based on the measured intracranial pressure.

15. The CSF drainage system of claim 13 further comprising a second valve configured to allow the first collection chamber to collect the CSF in a closed position and drain the collected CSF in the first collection chamber in an open position, and wherein the processor is further configured to open the second valve upon a determination that the intracranial pressure has reduced to a desired pressure value.

16. The CSF drainage system of claim 15 further comprising a second conduit having a proximal end and a distal end, the second conduit configured to receive the CSF from the first collection chamber from the proximal end and a second collection chamber coupled to the distal end of the second conduit and configured to collect the CSF transported by the second conduit, wherein the second valve is positioned on the second conduit.
